(19) United States
(12) Patent Application Publication
(10) Pub. No.: US 2025/0271413 A1
(43) Pub. Date: Aug. 28, 2025

(54) METHOD OF MEASURING THE CONTENT OF A CHEMICAL ELEMENT IN A COATING

(71) Applicant: NV Bekaert SA, Zwevegem (BE)

(72) Inventors: Degui XIA, Jiangyin (CN); Bart ALLAERT, Deinze (BE); Sebastiaan FORREZ, Menen (BE)

(73) Assignee: NV Bekaert SA, Zwevegem (BE)

(21) Appl. No.: 18/275,752

(22) PCT Filed: Feb. 25, 2022
(86) PCT No.: PCT/EP2022/054835

§ 371 Date: Aug. 3, 2023

(30) Foreign Application Priority Data
Feb. 26, 2021 (WO) PCT/CN2021/078074

(51) Int. Cl. G01N 33/2028; G01N 1/40; G01N 21/73

(52) U.S. Cl. CPC G01N 33/2028; G01N 1/4044; G01N 1/4077; G01N 21/73; G01N 2001/4094

(57) ABSTRACT

A method of measuring content of a first chemical element in a coating comprising the first element applied on a substrate also comprising the first element, the content of said the element being determined by measuring the ratio of the content of a second chemical element over the content of the first element in the coating and the ratio of the content of the second element over the content of the first element in the substrate, whereby the content of the first element both in the coating and in the substrate is different than the content of the second element both in the coating and in the substrate and the ratio of the content of a second element over the content of the first element in the coating is different from the ratio of the content of the second element over the content of the first element in the substrate.

### TECHNICAL FIELD

The present invention concerns a method of measuring the content of a first chemical element in a coating comprising said first chemical element applied on a substrate also comprising said first chemical element, whereby
the content of said first chemical element is determined by means of measuring the ratio of the content of a second chemical element over the content of said first chemical element in the coating as well as the ratio of the content of said second chemical element over the content of said first chemical element in the substrate,
whereby further
the content of the first chemical element both in the coating and in the substrate is different, preferably higher, than the content of the second chemical element both in the coating and in the substrate and further the ratio of the content of a second chemical element over the content of said first chemical element in the coating is different from the ratio of the content of said second chemical element over the content of said first chemical element in the substrate.

### BACKGROUND ART

In 2019 it is projected that about 2 billion steel cord reinforced tires for vehicles will be produced worldwide. The steel cord itself is made of steel filaments coated with a brass coating. The steel and the brass are relatively harmless to the environment and to human health.

However, in order to stabilize the adhesion between the skim compound and the steel cords, tire makers are adding cobalt based organic salts such as e.g. cobalt naphthenate, cobalt stearates or cobalt boron decanoate complexes to the rubber in addition to other additives such as carbon black, sulphur, accelerators, oils, antioxidants, activators, etc . . . . Some of these cobalt based organic salts are suspected to be carcinogenic and are more and more subject to restricted use.

It has therefore been considered for example in WO2020/156967 to remove the cobalt used in the tire altogether by using a cobalt free coating, especially a brass coating enriched with iron allows a good initial adhesion and adhesion retention in the common aging tests when using a completely cobalt free rubber.

Ensuring the quality of such coating is however difficult as both the coating and the underling steel substrate may comprise iron, so that measurements regarding the iron content of the coating may be hindered or biased by iron coming from the steel substrate.

There is accordingly the need for a new measuring method to easily, efficiently and reliably ensure the quality and/or composition of coatings such as those proposed in WO2020/156967, namely in coatings comprising a first chemical element applied on a substrate also comprising said first chemical element, so as to especially for example minimize any bias and/or influence coming from the substrate.

### DISCLOSURE OF INVENTION

The inventors set themselves the task to overcome the problems associated with the prior art. The main object of the invention is to allow an easy, efficient and/or reliable measurement of the content of a first chemical element in a coating comprising said first chemical element applied on a substrate also comprising said first chemical element. The present invention may thereby especially allow for example to minimize any bias and/or influence coming from the substrate.

The present invention thereby concerns a method of measuring the content of a first chemical element in a coating comprising said first chemical element applied on a substrate also comprising said first chemical element,
whereby the content of said first chemical element is determined by means of measuring the ratio of the content of a second chemical element over the content of said first chemical element in the coating as well as the ratio of the content of said second chemical element over the content of said first chemical element in the substrate,
whereby further the content of the first chemical element both in the coating and in the substrate is different, preferably higher, than the content of the second chemical element both in the coating and in the substrate and
further the ratio of the content of a second chemical element over the content of said first chemical element in the coating is different from the ratio of the content of said second chemical element over the content of said first chemical element in the substrate. Determined/determining or measured/measuring in the sense of the present invention may thereby also for example mean estimated, especially estimated based on known/supplied values and/or based on a regression run on several measured experimental values.

The problems described above thereby even increase when the coating and the substrate are subjected to a step or treatment, such as for example a wet wire drawing or dry wire drawing step, that may lead to or contribute to chemical elements, especially for example the first chemical element, preferably for example iron, migrating from the substrate to the coating or that may lead and/or contribute to increase the exposure of the substrate, which contains chemical elements and/or may lead or contribute to remove at least partially some of the applied coating, which comprises the first chemical element. In an embodiment of the present invention, the coating applied to a substrate may thus have been subjected to a wet wire drawing or dry wire drawing step before the measurement by the method according to the invention. This may thereby make measurements even more difficult, since on one hand chemical elements, especially the first chemical element, preferably for example iron, from the substrate may migrate partially to the coating and/or become increasingly exposed in such a step and/or since at the other hand some of the applied coating also comprising chemical elements, especially the first chemical element, preferably for example iron, may be at least partially removed. These different and/or antagonistic effects make it very difficult to foresee, estimate or measure the content of the first chemical element in the sense of the invention in the coating. According to the invention, the coating applied to a substrate may also be referred to as coated substrate.

In an embodiment of the present invention, the first chemical element may be iron and/or
the second chemical element may be selected from: manganese, chromium, silicon, vanadium, tungsten, nickel, molybdenum, aluminum, phosphor, sulfur, nitrogen or copper, preferably the second chemical element is manganese or silicon and/or the substrate may be steel.

In an embodiment of the present invention, the content of the first chemical element in the coating is determined by using the measured content of the first chemical element in the coating, the measured content of the second chemical element, and one or both ratios of content of the second chemical element over the content of the first chemical element selected from:
(E2/E1)_{deposited}: the ratio of content of second chemical element over the content of the first chemical element in the coating,
(E2/E1)_{substrate}: the ratio of content of second chemical element over the content of the first chemical element in the substrate,
and/or whereby the content of the first chemical element in the coating is determined by the following formula:

$$E {1}_{coating} = \frac{[ ( E {1}_{tot} \times {( \frac{E 2}{E 1} )}_{substrate} ) - E {2}_{tot} ]}{( {( \frac{E 2}{E 1} )}_{substrate} - {( \frac{E 2}{E 1} )}_{deposited} )}$$

whereby E1_{coating} is the relevant content of the first chemical element of the coating,
E1ₜₒₜ represents the total measured content of the first chemical element of the coating and the substrate, which may be obtained by adding up the contents as determined in all dissolution steps,
(E2/E1)_{substrate} represents the ratio of content of second chemical element over the content of the first chemical element in the substrate,
E2ₜₒₜ is the total measured content of the second chemical element of the coating and the substrate, which may be obtained by adding up the contents as determined in all dissolution steps and
(E2/E1)_{deposited} represents the ratio of content of second chemical element over the content of the first chemical element in the coating.

The ratio of content of second chemical element over the content of the first chemical element in the substrate (E2/E1)_{substrate} may be determined for example by measurements on the solution obtained from the last dissolution step. A dissolution step may thereby be a passivation step or a corrosion step. The corresponding ratio (E2/E1)_{substrate} may thereby also for example be determined by measurements carried out on the solution(s) obtained from one or more dissolution step(s) applied to the bare substrate without coating or estimated based on the substrate composition, especially for example as indicated by the supplier of the substrate. Similarly, the ratio of content of second chemical element over the content of the first chemical element in the coating (E2/E1)_{deposited} may be determined by measurements carried out on the solution(s) obtained from one or more dissolution step(s) applied to a corresponding half product, especially for example before a wet wire drawing step. A half product in the sense of the present invention may thereby especially be a product that comprises the substrate and the coating but which has not yet been subjected to a step and/or treatment that may lead or contribute to the diffusion of chemical elements, especially the first chemical element, from the substrate into the coating and/or may lead or contribute to increase the exposure of the substrate, which comprises the first chemical element and/or may lead or contribute to remove at least partially some of the applied coating, which comprises the first chemical element. As such, a half product may thus be a precursor to the coating applied on the substrate that is to be analyzed with the method according to the present invention. A half product may thereby be produced for example by:
a. providing a substrate;
b. coating said with a coating;
c. subjecting said coated intermediate substrate to a heat treatment.

The corresponding half product may thereby further be subjected to step that may lead or contribute to the diffusion of the first chemical element from the substrate into the coating and/or may lead or contribute to increase the exposure of the substrate, which comprises the first chemical element and/or may lead or contribute to remove at least partially some of the applied coating, which comprises the first chemical element. Such a step may especially for example be a wet wire drawing step or a dry drawing step, thereby obtaining the coated substrate according to the invention.

In a further embodiment of the invention, the iron content may be determined for example by performing at least one dissolution step and by using the equation:

$${Fe}_{coating} = \frac{[ ( {Fe}_{tot} \times {( \frac{Mn}{Fe} )}_{steel} ) - {Mn}_{tot} ]}{( {( \frac{Mn}{Fe} )}_{steel} - {( \frac{Mn}{Fe} )}_{deposited} )}$$

whereby further Fe_{coating} represents the iron content of the coating, Feₜₒₜ represents the total measured iron content of the steel substrate and the coating, which may be obtained by adding up the iron contents as determined in all dissolution steps,
(Mn/Fe)ₛₜₑₑₗ represents the ratio of the manganese (Mn) content over the iron (Fe) content of the steel substrate and may correspond to the following:
   (Mn/Fe)_{steel+brass dissolution}: the ratio of content of Mn over the content of the Fe measured in a first dissolution step, preferably a first passivation step, on a coating containing the same elements in same quantities but without first chemical element applied on the same substrate,
   (Mn/Fe)_{dissolution}: the ratio of Mn content over the content of Fe measured in a passivated or non-passivated environment (i.e. determined in a dissolution step carried out) on the bare substrate without coating, which may be determined by subjecting the substrate to one or more dissolution step(s) or alternatively which may be estimated based on the composition of the substrate, especially for example as provided by the supplier of the substrate.
whereby further Mnₜₒₜ represents the total measured manganese content of the steel substrate and the coating, which may be obtained by adding up the manganese contents as determined in all dissolution steps,
(Mn/Fe)_{deposited} represents the ratio of the manganese content of the coating over the iron content of the coating, which may for example be determined by measurements carried out on the solution(s) obtained from one or more dissolution step(s) applied to a corresponding half product, especially for example before a wet wire drawing step.

The total measured iron content of coating as part of Feₜₒₜ may be determined by carrying out one or more dissolution step(s) until the last measured ratio of the content of manganese over the content iron is within the range of ±50%, preferably ±40%, further preferred ±30%, further preferred ±20%, further preferred ±15%, of the ratio of the content of manganese over the content of iron of the steel substrate. The ratio of content of manganese over the content of iron in the steel substrate (Mn/Fe)ₛₜₑₑₗ may thereby be influenced by the sample preparation and especially the dissolution step(s) carried out and/or may preferably be determined by measurements on the solution obtained from the last dissolution step. The corresponding ratio (Mn/Fe)ₛₜₑₑₗ may thereby be determined by measurements or estimated based on the composition of the steel. Similarly, the ratio of content of manganese over the content of iron in the coating (Mn/Fe)_{deposited} may preferably be determined by measurements carried out on the solution(s) obtained from one or more dissolution step(s) applied to a corresponding half product, especially for example before a wet wire drawing step. The ratio of content of Mn over the content of the Fe measured in a first dissolution step, preferably a first passivation step, on a coating containing the same elements in same quantities but without first chemical element applied on the same substrate (Mn/Fe)_{steel+brass dissolution} may be determined by measurements on a solution obtained in a first passivation step carried out on a substrate with a brass coating containing the same elements in same quantities but without Fe applied on the same substrate. The ratio of Mn content over the content of Fe measured in a passivated or non-passivated environment (i.e. determined in a dissolution step carried out) on the bare substrate without coating (Mn/Fe)_{dissolution} may especially for example be obtained by using one or more dissolution steps carried out on the bare substrate without coating, possibly for example after complete dissolutions of the steel substrate by measurement on the solution(s) from the corresponding step(s), or be estimated based on the composition of the steel substrate, especially for example as provided by the supplier of the substrate.

A passivated environment may thereby especially for example be achieved by a dissolution step being a passivation step. On the other hand, a non-passivated environment may thereby especially for example be achieved by a dissolution step being a corrosion step.

In a further embodiment of the present invention, no further dissolution step may thus preferably carried out when the last measure ratio of the content of manganese over the content iron is within the range of ±50%, preferably ±40%, further preferred ±30%, further preferred ±20%, further preferred ±15%, of the ratio of the content of manganese over the content of iron of the steel substrate and/or
a further dissolution step is carried out when that is not the case.

In a further embodiment of the present invention, the iron content may be determined for example by subjecting the coated steel to one or preferably more than one, further preferred 1 to 10, even further-preferred 1 to 6 dissolution steps, even further preferred 1 dissolution step.

In a further embodiment of the present invention, the iron content may be determined for example performing n dissolution steps with n>1 and by using the equation:

$${Fe}_{coating} = \frac{[ ( {Fe}_{1} \cdot {( \frac{Mn}{Fe} )}_{steel + brass dissolution} ) - {Mn}_{1} ]}{( {( \frac{Mn}{Fe} )}_{steel + brass dissolution} - {( \frac{Mn}{Fe} )}_{deposited} )} + {\sum }_{i = 2}^{n} \frac{[ ( {Fe}_{( i )} \cdot {( \frac{Mn}{Fe} )}_{dissolution} ) - {Mn}_{( i )} ]}{( {( \frac{Mn}{Fe} )}_{dissolution} - {( \frac{Mn}{Fe} )}_{deposited} )}$$

whereby further Fe_{coating} represents the iron content of the plated coating, Fe₁ represents the iron amount determined in the first passivation step, (Mn/Fe)_{steel+brass dissolution} represents the ratio of content of Mn over the content of the Fe measured in a first step, preferably a first passivation step, on a coating containing the same elements in same quantities but without first chemical element applied on the same substrate,
Mn₁ represents the manganese amount determined in the first passivation step,
(Mn/Fe)_{deposited} represents the ratio of the manganese content of the coating over the iron content of the coating, which may be determined by measurements carried out on the solution(s) obtained from one or more dissolution step(s) applied to a corresponding half product, especially for example before a wet wire drawing step,
Fe₍ᵢ₎ represents the iron amount determined in dissolution step i, (Mn/Fe)_{dissolution} represents the ratio of Mn content over the content of Fe measured in a passivated or non-passivated environment (i.e. determined in a dissolution step carried out) on the bare substrate without coating, Mn₍ᵢ₎ represents the manganese amount determined in dissolution step i. The ratios mentioned above may again be determined by measurements, especially by measurements on corresponding solution, as explained herein, or estimated The (Mn/Fe)_{dissolution} may thereby especially for example be obtained by using one or more dissolution steps carried out on the bare substrate without coating, possibly for example after complete dissolutions of the steel substrate by measurement on the solution(s) from the corresponding step(s), or be estimated based on the composition of the steel substrate, especially for example as provided by the supplier of the substrate.

In a further embodiment of the present invention, values for Mn/Fe_{deposited}<0.1%, preferably <0.04%, preferably <0.02% or <0.01% may be for example be replaced by 0 in the formula(s) above.

In a further embodiment of the present invention, the iron content may be for example determined performing n dissolution steps with n>1 and by using the equation:

$${Fe}_{coating} = Fe 1 + {\sum }_{i = 2}^{n} \frac{[ ( {Fe}_{( i )} \cdot {( \frac{Mn}{Fe} )}_{* dissolution} ) - {Mn}_{( i )} ]}{( {( \frac{Mn}{Fe} )}_{dissolution} - {( \frac{Mn}{Fe} )}_{deposited} )}$$

whereby further Fe_{coating} represents the iron content of the coating,
Fe₁ represents the iron amount determined in a first passivation step, (Mn/Fe)_{deposited} represents the ratio of the manganese content of the coating over the iron content of the coating,
Fe₍ᵢ₎ represents the iron amount determined in dissolution step i, (Mn/Fe)_{dissolution} represents the ratio of Mn content over the content of Fe measured in a passivated or non-passivated environment (i.e. determined in a dissolution step carried out) on the bare substrate without coating, Mn₍ᵢ₎ represents the manganese content determined in dissolution step i. The method and formula above may thereby be preferably applicable when the duration of dissolution step(s) is short, especially for example between >10 and 40 minutes, and/or the number of dissolution step(s) is low, especially for example if one or two dissolution steps are carried out. The ratios mentioned above may again be determined by measurements, especially by measurements upon dissolution of the steel substrate, or estimated. (Mn/Fe)_{dissolution} may thereby especially for example be obtained by using one or more dissolution step(s) carried out on the bare substrate without coating, possibly for example after complete dissolutions of the steel substrate by measurement on the solution(s) from the corresponding step(s). (Mn/Fe)_{dissolution} may also be estimated based on the composition of the steel substrate, especially for example as provided by the supplier of the substrate.

The ratio of the manganese content of the coating over the iron content of the coating (Mn/Fe)_{deposited} may thereby especially be measured or determined on a half product, preferably before a wet wire drawing step. A half product may thereby be produced for example by:
d. providing a steel substrate;
e. electrolytically coating said steel substrate with copper, iron and zinc;
f. subjecting said copper-iron-zinc coated intermediate steel substrate to a heat treatment to diffuse the zinc into the copper at a temperature of at least 420° C. and below 530° C. resulting in an intermediate steel substrate with a brass coating enriched with iron particles.

The corresponding half product may thereby further be subjected for example to a wet wire drawing step thereby obtaining the coating applied on substrate to be analyzed using the method according to the invention. Moreover, bare substrate without coating may also mean especially for example bare substrate without any coating.

In a further embodiment of the present invention, the steel may be for example in the form of a steel cord and/or
the coating comprises brass and/or
the coating may be brass enriched with iron, preferably comprises copper, zinc and iron, further preferred the coating comprises on average >55 wt.-%, preferably >60 wt.-%, further preferred >62 wt.-%, even further preferred >63.0 wt.-% of copper, 1 to 10 wt.-% of iron, preferably 2 to 6 wt.-% of iron and the remainder of zinc.

In a further embodiment of the present invention, each dissolution step may be a passivation step or a corrosion step and/or
a passivation step may comprises especially for example using a stripping solution capable of stripping the coating and passivating the substrate, preferable using an ammonia/ammonium persulfate solution, further preferred a solution comprising 16 g of (NH₄)₂S₂O₈ and 120 mL NH₃ (25 wt.-%) brought to 1 liter by addition of water
and/or
a corrosion step may be using especially for example water and/or acidic solution. The coated steel substrate may thereby for example be subjected to a passivation step carried out under ultrasounds, preferably in a first dissolution step. Furthermore, a passivation step may be achieved by avoiding and/or reversing exposure to corrosive conditions. Corrosive conditions may thereby be conditions that lead to erosion of material by chemical reactions or processes. On the other hand, a corrosion step may be achieved by exposure to corrosive conditions. In some embodiments, acid or an acid solution may be added to the solution obtained from submitting the coated substrate to a passivation or a corrosion step to contribute to help with dissolving particles in such solution.

In a further embodiment of the present invention, the content of chemical elements, especially iron and manganese, is determined by inductive coupled plasma spectroscopy, preferably inductive coupled plasma optical emission spectroscopy or by inductive coupled plasma mass spectroscopy or by UV-visible spectroscopy or by a combination of liquid chromatography and mass spectroscopy or by x-ray fluorescence spectroscopy or by atomic absorption spectroscopy. Alternatively other suitable means or methods of measurements for the content of chemical elements may also be used.

In a further embodiment of the present invention, each dissolution step may be carried out for example under ultrasounds, preferably in a ultrasonic bath, for a duration of 5 to 480 minutes, preferably 10 to 90 minutes, further preferred 10 to 70 minutes, even further preferred >10 to 40 minutes and/or whereby each dissolution step may be carried out at a temperature between 0 to 80° C., preferably 5 to 60° C., further preferred 10 to 40° C. and/or
whereby each dissolution step may be carried out under ultrasounds, preferably in a ultrasonic bath, at a frequency of 20 to 100 kHz, further preferred 25 to 80 kHz.

In a further embodiment of the present invention, at least one passivation step is carried out before one or more controlled corrosion step. The first dissolution step may thereby preferably for example be a passivation step. In addition, one or more corrosion step(s) may be optional. Furthermore, all dissolution steps following at least one corrosion step may preferably be further corrosion steps.

In a further embodiment of the present invention, the coating may be for example non-homogeneous, preferably in that it allows access to the underlying steel and/comprises brass rich regions, preferably comprising >95 w.%, further preferred >97 w.% of brass, and/or comprises iron rich particles, preferably comprising >95 w.%, further preferred >97 w.% of iron.

### EXAMPLES

### Example 1

Sample A of steel substrates with a brass coating with average composition being 63.5 wt.-% Cu and the remainder being Zn as well as sample B of a steel cord with a coating having an average composition of 64 wt.-% of Cu and 4 wt.-% of Fe and the remainder being Zn applied to the half product were prepared. Samples A and B have been obtained by using a wet wire drawing step.

Samples A and B be were cut into pieces and 1.0 g of each sample was weighed on an electronic balance. The weighed samples were put into a test tube and 20 ml stripping solution was added into the tube. It is thereby important that the whole sample is submerged. If required, this may be achieved by selecting a test tube with an appropriate diameter.

1 L of stripping solution can thereby be prepared by adding 16 g ammonium persulfate into a beaker of 600 ml, and dissolve with 400 ml in ultrapure water. The 400 ml solution is then transferred quantitatively to a 1000 ml volume flask before 120 ml of an ammonia solution (25 wt.-%) are added to the flask. The flask is then further filled to the 1 L mark by ultrapure water to obtain the stripping solution.

The test tube is put in a stainless basket and then is subjected to a high performance lab ultrasonic cleaner bath (for example supplied by Fisherscientific part of Thermo Fisher Scientific under the designation Fisherbrand FB 11209) for 60 min. Parameters of ultrasonic cleaner include the following.
Frequency: 37 kHz
Power: 100%
Mode: pulse
The temperature may thereby be kept between 2° and 40° C.

After ultrasonic treatment, the resulting solution is transferred to a volume flask of 200 ml via a funnel. 5 ml 37 wt.-% HCl are then added into volume flask. Moreover, about 20 ml of ultrapure water is added into each test tube to rinse each sample. The rinsing water is also added into the volume flask. The rinsing process continues with further 20 ml portions until the rinsing water in the tube is visually clear (i.e. transparent). The sample is then taken out of test tube and 5 ml 37 wt.-% HCl is added into the test tube to rinse the wall of tube with acid. The resulting solution is also transferred into volume flask. Last, ultra pure water is added to reach the grade mark of 200 ml, if required.

The concentration of Fe (mg/l) and Mn (mg/l) in the solution within volume flask is then determined by ICP-OES (Inductive Coupled Plasma Optical Emission Spectroscopy).

Calculation of Fe (mg) in the coating per kg cord is carried out via equation 1, 2 and 3.

$${Fe}_{tot} = \frac{{Fe}_{flask} * Flask volume}{Sample weight} \qquad ( Equation 1 )$$

$${Mn}_{tot} = \frac{{Mn}_{flask} * Flask volume}{Sample weight} \qquad ( Equation 2 )$$

$${Fe}_{coating} = \frac{{Fe}_{tot} * {( \frac{Mn}{Fe} )}_{steel} - {Mn}_{tot}}{{( \frac{Mn}{Fe} )}_{steel} - {( \frac{Mn}{Fe} )}_{deposited}} \qquad ( Equation 3 )$$

Fe_{flask}: the Fe concentration in the volume flask as determined by ICP-OES, mg/l;
Mn_{flask}: the Mn concentration in the volume flask as determined by ICP-OES, mg/l;
Flask volume: volume of flask (200 ml), ml;
Sample weight: weight of sample, g;
Feₜₒₜ: the total measured Fe (mg) per kg cord calculated based on Equation 1, mg/kg;
Mnₜₒₜ: the total measured Mn (mg) per kg cord, mg/kg calculated based on Equation 2;
(Mn/Fe)ₛₜₑₑₗ: ratio of Mn to Fe in the steel substrate; in present case, namely 0.0052 for both samples A and B based on the known steel composition as provided by the supplier; (Mn/Fe)_{deposited}: ratio of Mn to Fe in the coating of the respective half products before a wet wire drawing step; namely respectively 0.0146 and 0.0006 for samples A and B;
Fe_{coating}: the weight (mg) of Fe in the coating per kg sample calculated based on Equation 3.

Table 1 shows the data the data of two samples A and B each measured 3 times. For sample A the theoretical Fe_{coating} should be 0 mg/kg and determinations indeed lead to values close to 0, indicating the method of the invention successfully distinguished between Fe from steel substrate and Fe from coating. On sample B, the determined Fe_{coating} of the three measurements shows very little variation and is significantly different from 0.

TABLE 1
result of ICP test and calculated Fe (mg/kg)

| | | Sample | | | | | |
|---|---|---|---|---|---|---|---|
| | | Weight | Fe_{flask} | Mn_{flask} | Fe_{coating+steel} | Mn_{coating+steel} | Fe_{coating} |
| Sample | Measurement | (g) | (mg/l) | (mg/l) | (mg/kg) | (mg/kg) | (mg/kg) |
| A | 1 | 1.0194 | 0.2059 | 0.0011 | 40.40 | 0.22 | 1.43 |
| | 2 | 1.0285 | 0.1850 | 0.0010 | 35.97 | 0.20 | 1.29 |
| | 3 | 1.0185 | 0.2485 | 0.0013 | 48.81 | 0.26 | 0.83 |
| B | 1 | 1.0221 | 0.9202 | 0.0015 | 180.06 | 0.29 | 140.91 |
| | 2 | 1.0019 | 0.9617 | 0.0016 | 191.98 | 0.32 | 146.99 |
| | 3 | 1.0719 | 1.0063 | 0.0017 | 187.76 | 0.31 | 143.44 |

### Example 2

Two pieces of sample C and D corresponding to 1 g+/−0.05 are cut and placed in test tubes. In parallel, multiple flasks (of 100 ml) are prepared for further use by adding 10 ml of HCl 37 wt.-% and 5 ml of internal standard solution, which may be for example a solution containing 50 ppm scandium in 3 v-% HNO₃ that may help to identify any possible measurement drifts).

The same high performance lab ultrasonic cleaner bath (see example 1 above) with the same parameters is used, whereby the bath temperature should be controlled between 3° and 40° C.

10 ml of the same stripping solution (see example 1 above) is added into the test tubes. The test tubes are treated in the ultrasonic bath for 20 minutes. Afterwards the solution is transferred quantitatively into a flask, the sample itself is thoroughly rinsed in the funnel with ultrapure water, whereby the rinsing water is also transferred to the flask. The samples C and D are then brought back into the test tube. The flask is left to thermostatize at room temperature and after enough time (about 30-40 minutes) has passed, filled up to the mark with ultrapure water (leading to solutions C and D in Table 2 below).

Immediately after transferring the previous sample solution into a flask, sample C is once again treated with 10 ml of stripping solution, ensuring no or minimal exposure to a corrosive environment, to carry out a dissolution step being a passivation step. The test tube is then again placed into the ultrasonic bath and subjected to an ultrasound treatment for 20 minutes. Afterwards the solution is transferred quantitatively into a new flask, the sample itself is thoroughly rinsed in the funnel with ultrapure water. The sample is then brought back into the sample container. The flask is left to thermostatize at room temperature and after enough time has passed, filled up to the mark with ultrapure water. This procedure is then repeated 2 more times on sample C to carry out a total of 3 dissolution steps being passivation steps (leading to solutions C1 to C3 in Table 2 below).

On the other hand for sample D, immediately after transferring the previous sample solution into a flask as per paragraph [0048], sample D is treated with 5 ml of ultrapure water. The sample is then allowed to rest for 1 hour. Afterwards 10 ml of stripping solution is added to carry out a dissolution step being a corrosion step. The test tube is then again placed into the ultrasonic bath and subjected to an ultrasound treatment for 20 minutes. Afterwards the solution is transferred quantitatively into a new flask, the sample itself is thoroughly rinsed in the funnel with ultrapure water, whereby the rinsing water is also transferred to the flask. The cord sample is then brought back into the sample test tube. The flask is left to thermostatize and after enough time has passed, filled up to the mark with ultrapure water. This procedure is then repeated 2 more times on sample D to carry out a total of 3 dissolution steps being corrosion steps (leading to solutions D1 to D3 below).

Results determined by ICP-OES on the solutions for sample C are show in Table 2 below.

TABLE 2

| | Fe | Mn |
|---|---|---|
| Solutions | (ppm/g) | (ppt/g) |
| C | 1.115128 | 2370.562 |
| C1 | 0.239258 | 643.2477 |
| C2 | 0.073293 | 209.7134 |
| C3 | 0.059897 | 273.617 |

Fe_{coating} can thereby be determined as follows:

$${Fe}_{coating} = \frac{[ ( {Fe}_{1} \cdot {( \frac{Mn}{Fe} )}_{steel + brass dissolution} ) - {Mn}_{1} ]}{( {( \frac{Mn}{Fe} )}_{steel + brass dissolution} - {( \frac{Mn}{Fe} )}_{deposited} )} + {\sum }_{i = 2}^{n} \frac{[ ( {Fe}_{( i )} \cdot {( \frac{Mn}{Fe} )}_{dissolution} ) - {Mn}_{( i )} ]}{( {( \frac{Mn}{Fe} )}_{dissolution} - {( \frac{Mn}{Fe} )}_{deposited} )}$$

$${Fe}_{coating} = \frac{[ ( 1 , 12.0 , 83 \% ) - 0 , 0024 ]}{( 0 , 83 \% - 0 , 07 \% )} + \frac{[ ( 0 , 37.1 , 13 \% ) - 0 , 0011 ]}{( 1 , 13 \% - 0 , 07 \% )}$$

$$\text{.1000} / 10 to get mg / kg ( g to kg and 100 mL solution )$$

$${Fe}_{coating} = 119 , 7 mg / kg$$

Fe_{coating} thereby represents the iron content of the plated coating. Fe₁ represents the iron amount determined by ICP-OES in the first passivation step and thus here on solution C.

(Mn/Fe)_{steel+brass dissolution} represents the ratio of content of Mn over the content of the Fe in a brass coating containing the same elements in same quantities but without Fe applied on the same substrate estimated based on Equation 4 below by ICP-OES on a solution obtained in a first passivation step as described in [0048],

Mn₁ represents the manganese amount determined by ICP-OES in the first passivation step and thus here again on solution C.

(Mn/Fe)_{deposited} represents the ratio of the manganese content of the coating over the iron content of the coating determined by ICP-OES on the solution obtained by carrying out one passivation step as described above in [0048] on the corresponding half product before the wet wire drawing step.

Fe₍ᵢ₎ represents the iron amount determined in dissolution step i and corresponds to the values determined by ICP-OES for the solutions C1 to C3, which may be added up in view of the formula above.

(Mn/Fe)_{dissolution} represents the ratio of Mn content over the content of Fe measured in a passivated or non-passivated environment (i.e. determined in a dissolution step carried out) on the bare substrate without coating and is determined for sample C as indicated in Table 4 below. Alternatively, (Mn/Fe)_{dissolution} can also be estimated based on steel composition of the steel substrate.

Mn₍ᵢ₎ represents the manganese amount determined in passivation step i and again corresponds to the values determined by ICP-OES for the solutions C1 to C3, which may be added up in view of the formula above.

Alternatively, Fe_{coating} can also be determined as follows:

$${Fe}_{coating} = Fe 1 + {\sum }_{i = 2}^{n} \frac{[ ( {Fe}_{( i )} \cdot {( \frac{Mn}{Fe} )}_{dissolution} ) - {Mn}_{( i )} ]}{( {( \frac{Mn}{Fe} )}_{dissolution} - {( \frac{Mn}{Fe} )}_{deposited} )}$$

$${Fe}_{coating} = 1 , 12 + \frac{[ ( 0 , 37.1 , 13 \% ) - 0 , 0011 ]}{( 1 , 13 \% - 0 , 07 \% )}$$

$$\text{.1000} / 10 to get mg / kg ( g to kg and 100 mL solution )$$

$${Fe}_{coating} = 140 , 6 mg / kg$$

Fe_{coating} represents the iron content of the coating.

Fe₁ represents the iron amount determined by ICP-OES in the first passivation step and thus here on solution C.

(Mn/Fe)_{deposited} represents the ratio of the manganese content of the coating over the iron content of the coating determined by ICP-OES on the solution obtained by carrying out one passivation step as described above in [0048] on the corresponding half product before the wet wire drawing step.

Fe₍ᵢ₎ represents the iron amount determined in dissolution step i and corresponds to the values determined by ICP-OES for the solutions C1 to C3, which may be added up in view of the formula above.

(Mn/Fe)_{dissolution} represents the ratio of Mn content over the content of Fe measured in a passivated or non-passivated environment (i.e. determined in a dissolution step carried out) on the bare substrate without coating and is determined for sample C as indicated in Table 4 below. Alternatively, (Mn/Fe)_{dissolution} can also be estimated based on steel composition of the steel substrate.

Mn₍ᵢ₎ represents the manganese amount determined in passivation step i and corresponds to the values determined by ICP-OES for the solutions C1 to C3, which may be added up in view of the formula above.

Results determined by ICP-OES on the solutions for sample D are show in Table 3 below.

TABLE 3

| | Fe | Mn |
|---|---|---|
| Solutions | (ppm/g) | (ppt/g) |
| D | 0.916177106 | 2185.813862 |
| D1 | 1.183546044 | 5750.215983 |
| D2 | 0.975597879 | 4801.20754 |
| D3 | 0.452410171 | 2393.726684 |

Fe_{coating} can thereby be determined as follows:

$${Fe}_{coating} = \frac{[ ( {Fe}_{1} \cdot {( \frac{Mn}{Fe} )}_{steel + brass dissolution} ) - {Mn}_{1} ]}{( {( \frac{Mn}{Fe} )}_{steel + brass dissolution} - {( \frac{Mn}{Fe} )}_{deposited} )} + {\sum }_{i = 2}^{n} \frac{[ ( {Fe}_{( i )} \cdot {( \frac{Mn}{Fe} )}_{* dissolution} ) - {Mn}_{( i )} ]}{( {( \frac{Mn}{Fe} )}_{* dissolution} - {( \frac{Mn}{Fe} )}_{deposited} )}$$

$${Fe}_{coating} = \frac{[ ( 0 , 92.0 , 83 \% ) - 0 , 0022 ]}{( 0 , 83 \% - 0 , 07 \% )} + \frac{[ ( 2 , 61.1 , 13 \% ) - 0 , 013 ]}{( 0 , 60 \% - 0 , 07 \% )}$$

$$\text{.1000} / 10 to get mg / kg ( g to kg and 100 mL solution )$$

$${Fe}_{coating} = 122 , 7 mg / kg$$

Fe_{coating} thereby represents the iron content of the plated coating.

Fe₁ represents the iron amount determined by ICP-OES in the first passivation step and thus here on solution D.

(Mn/Fe)_{steel+brass dissolution} represents the ratio of content of Mn over the content of the Fe in a brass coating containing the same elements in same quantities but without Fe applied on the same substrate estimated based on Equation 4 below by ICP-OES on a solution obtained in a first passivation step as described in [0048],

Mn₁ represents the manganese amount determined by ICP-OES in the first passivation step and thus here again on solution D.

(Mn/Fe)_{deposited} represents the ratio of the manganese content of the coating over the iron content of the coating determined by ICP-OES on the solution obtained by carrying out one passivation step as described above in [0048] on the corresponding half product before the wet wire drawing step.

Fe₍ᵢ₎ represents the iron amount determined in dissolution step i and corresponds to the values determined by ICP-OES for the solutions D1 to D3, which may be added up in view of the formula above.

(Mn/Fe)_{dissolution} represents the ratio of Mn content over the content of Fe measured in a passivated or non-passivated environment (i.e. determined in a dissolution step carried out) on the bare substrate without coating and is determined for sample D as indicated in Table 4 below. Alternatively, (Mn/Fe)_{dissolution} can also be estimated based on steel composition of the steel substrate.

Mn₍ᵢ₎ represents the manganese amount determined in passivation step i and corresponds to the values determined by ICP-OES for the solutions D1 to D3, which may be added up in view of the formula above.

Alternatively, Fe_{coating} can also be determined as follows:

$${Fe}_{coating} = Fe 1 + {\sum }_{i = 2}^{n} \frac{[ ( {Fe}_{( i )} \cdot {( \frac{Mn}{Fe} )}_{dissolution} ) - {Mn}_{( i )} ]}{( {( \frac{Mn}{Fe} )}_{dissolution} - {( \frac{Mn}{Fe} )}_{deposited} )}$$

$${Fe}_{coating} = 0 , 92 + \frac{[ ( 2 , 61.113 \% ) - 0 , 013 ]}{( 0 , 60 \% - 0 , 07 \% )}$$

$$\text{.1000} / 10 to get mg / kg ( g to kg and 100 mL solution )$$

$${Fe}_{coating} = 143 , 0 mg / kg$$

Fe_{coating} represents the iron content of the coating.

Fe₁ represents the iron amount determined by ICP-OES in the first passivation step and thus here on solution D.

(Mn/Fe)_{deposited} represents the ratio of the manganese content of the coating over the iron content of the coating determined by ICP-OES on the solution obtained by carrying out one passivation step as described above in [0048] on the corresponding half product before the wet wire drawing step.

Fe₍ᵢ₎ represents the iron amount determined in dissolution step i and corresponds to the values determined by ICP-OES for the solutions D1 to D3, which may be added up in view of the formula above.

(Mn/Fe)_{dissolution} represents the ratio of Mn content over the content of Fe measured in a passivated or non-passivated environment (i.e. determined in a dissolution step carried out) on the bare substrate without coating and is determined for sample D as indicated in Table 4 below. Alternatively, (Mn/Fe)_{dissolution} can also be estimated based on steel composition of the steel substrate.

Mn₍ᵢ₎ represents the manganese amount determined in passivation step i and corresponds to the values determined by ICP-OES for the solutions D1 to D3, which may be added up in view of the formula above.

Whereby some of the ratios were estimated or determined as indicated in Table 4 below.

TABLE 4

| Value | Determined/Estimated |
|---|---|
| ${( \frac{Mn}{Fe} )}_{steel + brass dissolution} = 0.83 \%$ | Estimated using Equation 4 below |
| ${( \frac{Mn}{Fe} )}_{deposited} = 0.07 \%$ | Determined on the corresponding half product before the wet wire drawing step |
| ${( \frac{Mn}{Fe} )}_{dissolution} C = 1.13 \%$ | Determined by ICP-OES on a solution obtained by carrying out a passivation step as per [0048] |
| | but on the bare steel substrate |
| | without coating |
| ${( \frac{Mn}{Fe} )}_{dissolution} D = 0.60 \%$ | Determined by ICP-OES on a solution obtained by carrying out a corrosion step as per [0050] but |
| | on the bare steel substrate |
| | without coating after a passivation |
| | step as per [0048] |

$${( \frac{Mn}{Fe} )}_{steel + brass dissolution} = - 0 , 001101 + 1 , 592 \times {( \frac{Mn}{Fe} )}_{dissolution} D \qquad Equation 4$$

Equation 4 is thereby estimated based on a regression run on several values obtained by ICP-OES for solutions obtained by carrying at least two dissolution steps with at least one corrosion step on brass coating containing the same elements in same quantities as C and/or D (but without Fe) applied on steel substrates.

1-15. (canceled)

16. A method of measuring the content of a first chemical element in a coating comprising said first chemical element applied on a substrate also comprising said first chemical element,
whereby the content of said first chemical element is determined by means of measuring the ratio of the content of a second chemical element over the content of said first chemical element in the coating as well as the ratio of the content of said second chemical element over the content of said first chemical element in the substrate,
whereby further
the content of the first chemical element both in the coating and in the substrate is different than the content of the second chemical element both in the coating and in the substrate and
further the ratio of the content of a second chemical element over the content of said first chemical element in the coating is different from the ratio of the content of said second chemical element over the content of said first chemical element in the substrate.

17. The method according to claim 16,
whereby the content of the first chemical element both in the coating and in the substrate is higher than the content of the second chemical element both in the coating and in the substrate and/or whereby the first chemical element is iron and/or
whereby the second chemical element may be selected from: manganese, chromium, silicon, vanadium, tungsten, nickel, molybdenum, aluminum, phosphor, sulfur, nitrogen or copper, preferably the second chemical element is manganese or silicon and/or whereby the substrate is steel.

18. The method according to claim 16,
whereby the content of the first chemical element in the coating is determined by using the measured content of the first chemical element in the coating, the measured content of the second chemical element, and one or both ratios of content of the second chemical element over the content of the first chemical element selected from:
(E2/E1)_{deposited}: the ratio of content of second chemical element over the content of the first chemical element in the coating,
(E2/E1)_{substrate}: the ratio of content of second chemical element over the content of the first chemical element in the substrate,
and/or whereby the content of the first chemical element in the coating is determined by the following formula:
$$E {1}_{coating} = \frac{[ ( E {1}_{tot} \times {( \frac{E 2}{E 1} )}_{substrate} ) - E {2}_{tot} ]}{( {( \frac{E 2}{E 1} )}_{substrate} - {( \frac{E 2}{E 1} )}_{deposited} )}$$
whereby E1_{coating} is the relevant content of the first chemical element of the coating, E1ₜₒₜ represents the total measured content of the first chemical element of the coating and the substrate after a sample preparation,
(E2/E1)_{substrate} represents the ratio of content of second chemical element over the content of the first chemical element in the substrate,
E2ₜₒₜ is the total measured content of the second chemical element of the coating and the substrate after a sample preparation and
(E2/E1)_{deposited} represents the ratio of content of second chemical element over the content of the first chemical element in the coating.

19. The method according to claim 16,
whereby the iron content is determined by performing at least one dissolution step and by using the equation:
$${Fe}_{coating} = \frac{[ ( {Fe}_{tot} \times {( \frac{Mn}{Fe} )}_{steel} ) - {Mn}_{tot} ]}{( {( \frac{Mn}{Fe} )}_{steel} - {( \frac{Mn}{Fe} )}_{deposited} )}$$
whereby further Fe_{coating} represents the iron content of the coating,
Feₜₒₜ represents the total measured iron content of the steel substrate and the coating after a sample preparation,
(Mn/Fe)ₛₜₑₑₗ represents the ratio of the manganese content over the iron content of the steel substrate and may correspond to the following:
(Mn/Fe)_{steel+brass dissolution}: the ratio of content of Mn over the content of the Fe measured in a first dissolution step, preferably a first passivation step, on a coating containing the same elements in same quantities but without first chemical element applied on the same substrate,
(Mn/Fe)_{dissolution}: the ratio of Mn content over the content of Fe measured in a passivated or non-passivated environment (i.e. determined in a dissolution step carried out) on the bare substrate without coating,
whereby further Mnₜₒₜ represents the total measured manganese content of the steel substrate and the coating after a sample preparation,
(Mn/Fe)_{deposited} represents the ratio of the manganese content of the coating over the iron content of the coating.

20. The method according to claim 16,
whereby the iron content is determined by subjecting the coated steel to one or preferably more than one, further preferred 1 to 10, even further preferred 1 to 6 dissolution steps, even further preferred 1 dissolution step.

21. The method according to claim 16,
whereby the iron content is determined performing n dissolution steps with n>1 and by using the equation:
$${Fe}_{coating} = \frac{[ ( {Fe}_{1} \cdot {( \frac{Mn}{Fe} )}_{steel + brass dissolution} ) - {Mn}_{1} ]}{( {( \frac{Mn}{Fe} )}_{steel + brass dissolution} - {( \frac{Mn}{Fe} )}_{deposited} )} + {\sum }_{i = 2}^{n} \frac{[ ( {Fe}_{( i )} \cdot {( \frac{Mn}{Fe} )}_{dissolution} ) - {Mn}_{( i )} ]}{( {( \frac{Mn}{Fe} )}_{dissolution} - {( \frac{Mn}{Fe} )}_{deposited} )}$$
whereby further Fe_{coating} represents the iron content of the plated coating,
Fe₁ represents the iron amount determined in the first passivation step,
(Mn/Fe)_{steel+brass dissolution} represents the ratio of content of Mn over the content of the Fe measured in a first step, preferably a first passivation step, on a coating containing the same elements in same quantities but without first chemical element applied on the same substrate,
Mn₁ represents the manganese amount determined in the first passivation step,
(Mn/Fe)_{deposited} represents the ratio of the manganese content of the coating over the iron content of the coating,
Fe₍ᵢ₎ represents the iron amount determined in dissolution step i,
(Mn/Fe)_{dissolution} represents the ratio of Mn content over the content of Fe measured in a passivated or non-passivated environment (i.e. determined in a dissolution step carried out) on the bare substrate without coating,
Mn₍ᵢ₎ represents the manganese amount determined in passivation step i.

22. The method according to claim 16, where values for Mn/Fe_{deposited}<0.1%, preferably <0.04%, preferably <0.02% or <0.01% can be replaced by 0 in the formula(s).

23. The method according to claim 16,
whereby the iron content is determined performing n dissolution steps with n>1 and by using the equation:
$${Fe}_{coating} = Fe 1 + {\sum }_{i = 2}^{n} \frac{[ ( {Fe}_{( i )} \cdot {( \frac{Mn}{Fe} )}_{dissolution} ) - {Mn}_{( i )} ]}{( {( \frac{Mn}{Fe} )}_{dissolution} - {( \frac{Mn}{Fe} )}_{deposited} )}$$
whereby further Fe_{coating} represents the iron content of the coating,
Fe₁ represents the iron amount determined in a first passivation step,
(Mn/Fe)_{deposited} represents the ratio of the manganese content of the coating over the iron content of the coating,
Fe₍ᵢ₎ represents the iron amount determined in dissolution step i,
(Mn/Fe)_{dissolution} represents the ratio of Mn content over the content of Fe measured in a passivated or non-passivated environment (i.e. determined in a dissolution step carried out) on the bare substrate without coating,
Mn₍ᵢ₎ represents the manganese content determined in dissolution step i.

24. The method according to claim 16,
whereby the steel is in the form of a steel cord, and/or
the coating comprises brass, and/or
the coating may be brass enriched with iron, preferably comprises copper, zinc and iron, further preferred the coating comprises on average >55 wt.-%, preferably >60 wt.-%, further preferred >62 wt.-%, even further preferred >63.0 wt.-% of copper, 1 to 10 wt.-% of iron, preferably 2 to 6 wt.-% of iron and the remainder of zinc.

25. The method according to claim 16,
whereby each dissolution step may be a passivation step or a corrosion step, and/or
whereby a passivation step may comprises using a stripping solution capable of stripping the coating and passivating the substrate, preferable using an ammonia/ammonium persulfate solution, further preferred a solution comprising 16 g of (NH₄)₂S₂O₈ and 120 mL NH₃ (25 wt.-%) brought to 1 liter by addition of water,
and/or
whereby a corrosion step may be using water and/or acidic solution.

26. The method according to claim 16,
whereby the content of chemical elements, especially iron and manganese, is determined by inductive coupled plasma spectroscopy, preferably inductive coupled plasma optical emission spectroscopy or by inductive coupled plasma mass spectroscopy or by UV-visible spectroscopy or by a combination of liquid chromatography and mass spectroscopy or by X-ray fluorescence spectroscopy or by atomic absorption spectroscopy.

27. The method according to claim 16,
whereby each dissolution step is carried out under ultrasounds, preferably in an ultrasonic bath, for a duration of 5 to 480 minutes, preferably 10 to 90 minutes, further preferred 10 to 70 minutes, even further preferred >10 to 40 minutes, and/or
whereby each dissolution step is carried out at a temperature between 0 to 80° C., preferably 5 to 60° C., further preferred 10 to 40° C., and/or
whereby each dissolution step is carried out under ultrasounds, preferably in a ultrasonic bath, at a frequency of 20 to 100 kHz, further preferred 25 to 80 kHz.

28. The method according to claim 16,
whereby at least one passivation step is carried out before one or more controlled corrosion step.

29. The method according to claim 16,
whereby the coating is non-homogeneous, preferably in that it allows access to the underlying steel and/comprises brass rich regions, preferably comprising >95 w.%, further preferred >97 w.% of brass, and/or comprises iron rich particles, preferably comprising >95 w.%, further preferred >97 w.% of iron.

30. The method according to claim 16,
whereby no further dissolution step is carried out when the last measure ratio of the content of manganese over the content iron is within the range of ±50%, preferably ±40%, further preferred ±30%, further preferred ±20%, further preferred ±15%, of the ratio of the content of manganese over the content of iron of the steel substrate, and/or
whereby a further dissolution step is carried out when that is not the case.
